(19) United States
(12) Patent Application Publication
(10) Pub. No.: US 2018/0092554 A1
(43) Pub. Date: Apr. 5, 2018

(54) WEARABLE AND UNOBTRUSIVE MULTI-SENSOR ARRAY AND METHOD FOR PULSE WAVE VELOCITY IMAGING

(71) Applicant: THE CHINESE UNIVERSITY OF HONG KONG, HONG KONG (CN)

(72) Inventors: Yuanting ZHANG, HONG KONG (CN); Hon Ki TSANG, HONG KONG (CN); Ni ZHAO, Guangdong (CN); Xiaorong DING, Sichuan Province (CN); Wenxuan DAI, Zhejiang (CN); Jing LIU, Hubei (CN)

(21) Appl. No.: 15/282,689

(22) Filed: Sep. 30, 2016

(51) Int. Cl. A61B 5/024; A61B 5/00; A61B 5/0205; A61B 5/1455

(52) U.S. Cl. CPC A61B 5/02438; A61B 5/0022; A61B 5/0205; A61B 5/14552; A61B 5/0816; A61B 5/6892; A61B 5/6805; A61B 5/024; A61B 5/6804

(57) ABSTRACT

Novel and advantageous systems, devices, and methods for pulse wave velocity (PWV) imaging that enable precise central blood pressure (BP) estimation are provided. A multi-sensor array can provide two-dimensional PWV, thereby achieving the PWV imaging and precise central BP estimation. The multi-sensor array can be integrated in daily objects, such as clothing or a bed, which results in noninvasive, continuous, low-cost monitoring of BP that is easy to use and does not require a medical professional. Electrocardiogram imaging, photoplethysmogram imaging, and monitoring for other physiological parameters such as heart rate, SpO₂, BP, and respiration, can also be achieved.

### FIELD OF THE INVENTION

Embodiments of the present invention relate to wearable multi-sensor arrays for unobtrusive and continuous health status monitoring. Embodiments relate further to methods for generating pulse wave velocity imaging and thereby estimating central blood pressure. Still further, embodiments of the present invention relate to multi-sensor array designs and methods for estimating blood pressure.

### BACKGROUND

Pulse wave velocity (PWV) is an important physiological parameter for evaluating arterial stiffness, which is an independent predictor of cardiovascular morbidity and mortality, as well as a potential indicator to track blood pressure changes. PWV is typically assessed by the arrival time of a pressure wave propagating through the arterial tree for a certain distance between the proximal and distal arterial sites. Pulse transit time (PTT) has a reciprocal relationship with PWV, and can be obtained from two pulse signals. PTT can be used for BP estimation without the need for a cuff. The estimated BP can be obtained by calibration with a cuff-based BP device that measures brachial BP. Despite the fact that cuff-less BP measuring is noninvasive and continuous, PTT-based methods can only provide peripheral BP. However, emerging evidence suggests that central blood pressure is more closely related to future cardiovascular events than is brachial pressure. There are several methods available for assessing central blood pressure. The invasive cardiac catheterization is the gold standard for measurement of central BP, but it is highly invasive and limited to only clinical settings. In addition, noninvasive methods can be used, such as the applanation tonometry of the carotid artery, applanation tonometry of the radial artery, and cuff-based oscillometry at the brachial artery. However, these methods require skilled medical professionals for operation and/or applanation against the artery and the occluded inflation cuff. Thus, they are not suitable for long-term continuous and unobtrusive applications.

Current techniques for PWV imaging include ultrasound methods and magnetic resonance imaging (MRI). These techniques provide good resolution, but they have several disadvantages, including bulky size, the requirement of skilled medical staff for operation, and being limited to clinical usage. Therefore, they are not suitable for continuous long-term applications.

### BRIEF SUMMARY

Embodiments of the subject invention provide novel and advantageous systems, devices, and methods for pulse wave velocity (PWV) imaging that enable precise central blood pressure (BP) estimation. In many embodiments, a multi-sensor array can provide two-dimensional PWV, thereby achieving the PWV imaging and precise central BP estimation. The multi-sensor array can be integrated in daily objects (e.g., clothing or a bed), which results in noninvasive, unobtrusive, continuous, low-cost monitoring of BP that is easy to use and does not require a medical professional. Embodiments are therefore suitable for long-term monitoring for cardiovascular disease. In addition, embodiments can also provide electrocardiogram (ECG) imaging, photoplethysmogram (PPG) imaging, and monitoring for other physiological parameters such as heart rate, SpO₂, BP, and respiration.

In one embodiment, a system for PWV imaging can comprise: a plurality of sensor units, each sensor unit including at least one electrical sensor for detecting ECG signals of a user and at least one optical sensor for detecting PPG signals of said user, wherein each optical sensor includes a light source emitting light at a first wavelength and a photodetector, a front-end circuit in operable communication with the plurality of sensor units, the front-end circuit comprising at least one amplifier and filter circuit to amplify and filter the ECG and PPG signals and at least one analog-to-digital converter to digitize the filtered ECG and PPG signals; and at least one processor in operable communication with the front-end circuit to calculate pulse transmit time (PTT) from the ECG and PPG signals, thereby generating PWV imaging. The plurality of sensor units and the front-end circuit can be provided on an everyday object, such as a wearable garment (e.g., a shirt, a vest, or pants) or a bed.

### BRIEF DESCRIPTION OF THE DRAWINGS

FIG. 1 shows a schematic view of a multi-sensor array shirt according to an embodiment of the subject invention.

FIG. 2 shows a block diagram of a system according to an embodiment of the subject invention.

FIG. 3 shows a schematic view of the shirt of FIG. 1, equipped with a sensor pair array.

FIG. 4 shows a schematic view of the sensor pair array of FIG. 3.

FIG. 5 shows a schematic view of the sensor pair array of FIG. 4 measuring the electrocardiogram (ECG) and photoplethysmogram (PPG) signals of the user at different body sites.

FIG. 6 shows a schematic view of a sleeping bed mounted with a multi-sensor array according to an embodiment of the subject invention.

FIG. 7 shows a schematic view of a PPG body sensor network to improve the accuracy of central BP estimation based on a PTT method, according to an embodiment of the subject invention.

FIG. 8 shows a block diagram of a remote terminal displaying the pulse wave velocity imaging, according to an embodiment of the subject invention.

### DETAILED DISCLOSURE OF THE INVENTION

Embodiments of the subject invention provide novel and advantageous systems, devices, and methods for pulse wave velocity (PWV) imaging that enable precise central blood pressure (BP) estimation. In many embodiments, a multi-sensor array can provide two-dimensional PWV, thereby achieving the PWV imaging and precise central BP estimation. The multi-sensor array can be integrated in daily objects (e.g., clothing or a bed), which results in noninvasive, unobtrusive, continuous, low-cost monitoring of BP that is easy to use and does not require a medical professional. Embodiments are therefore suitable for long-term monitoring for cardiovascular disease. In addition, embodiments can also provide electrocardiogram (ECG) imaging, photoplethysmogram (PPG) imaging, and monitoring for other physiological parameters such as heart rate, SpO₂, BP, and respiration.

PWV is an important physiological parameter for evaluating arterial stiffness and tracking BP changes, as well as an independent predictor of cardiovascular morbidity and mortality. Current techniques for PWV measurement are through detecting arterial pulses at two different peripheral sites, so the measured PWV is one-dimensional and provides limited information about the cardiovascular system. In addition, existing cuff-less BP measurement methods based on PWV recording are available only for peripheral BP estimation. Central BP (CBP) is a better indicator for future cardiovascular events as compared to peripheral BP pressure (e.g., brachial pressure), but existing cuff-less BP based on PWV recording methods based on PWV recording cannot determine or estimate CBP.

Embodiments of the subject invention include multi-sensor layouts (e.g., multi-sensor arrays) that enable two-dimensional PWV measurements, as well as precise CBP estimation. A multi-sensor layout can include a plurality of sensor pairs, with each sensor node including one or more ECG electrodes and one or more PPG optical sensors. These sensor pairs provide PWV imaging and lead to CBP estimation, in addition of physiological monitoring of ECG imaging, PPG imaging, and physiological parameters including but not necessarily limited to heart rate, SpO2, BP, and respiration. Each sensor element can be utilized as a pixel and can be assembled in various ways to achieve different resolutions that adapt to different applications. A multi-sensor layout can be incorporated into an everyday object, such as a wearable garment (e.g., a shirt, a vest, pants) or a bed, such that noninvasive and continuous monitoring can be performed without the need for a medical professional.

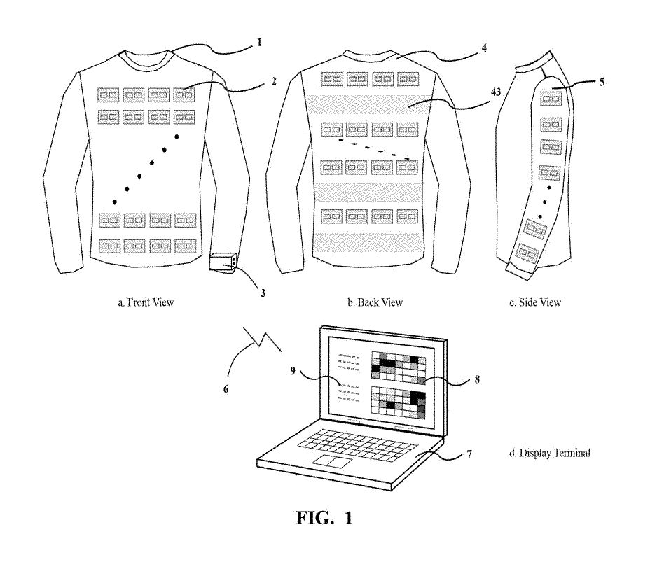

FIG. 1 shows a schematic view of a PWV imaging system including shirt having a multi-sensor array according to an embodiment of the subject invention. Referring to FIG. 1, the multi-sensor array can be incorporated into (e.g., tailored on) the front, the back 4, and/or a side 5 of the shirt 1. The shirt can be made of any known material for garments and can include an integrated sensing element 2 and/or an e-textile electrode 43. The configuration of the array can be designed according to the requirements of comfort, accuracy, and spatial resolution. During a measurement, the shirt positioned up close to the body to contact the user's skin can measure the PPG and ECG waveforms. The system can also include a front-end circuit 3, which can include a power source (e.g., a battery source, such as one or more (rechargeable) AA batteries), and amplifier and filter circuit, an analog-to-digit converter, and an output transmitter. The output transmitter can be configured to transmit wirelessly and can be, for example, a wireless Bluetooth transceiver, though embodiments are not limited thereto. The power source (e.g., one or more batteries) can support the running of all the electrical components on the garment. After being transduced by the multi-sensor array, the signals can first be sent to the amplifier and filter circuit and then to the analog-to-digit converter that digitizes the waveform (e.g., at 1 kilohertz (kHz) with, for example, 16-bit resolution). After digitalization, the data can be transmitted 6 (e.g., by a short-range wireless Bluetooth transceiver 6) to an external signal processing box for further processing. The system can also include a display terminal 7 that communicates with the sensor array device (e.g., sensor array shirt) in real time. The terminal can process the received signals and display PWV images 8 and estimated CBP values 9 on the screen.

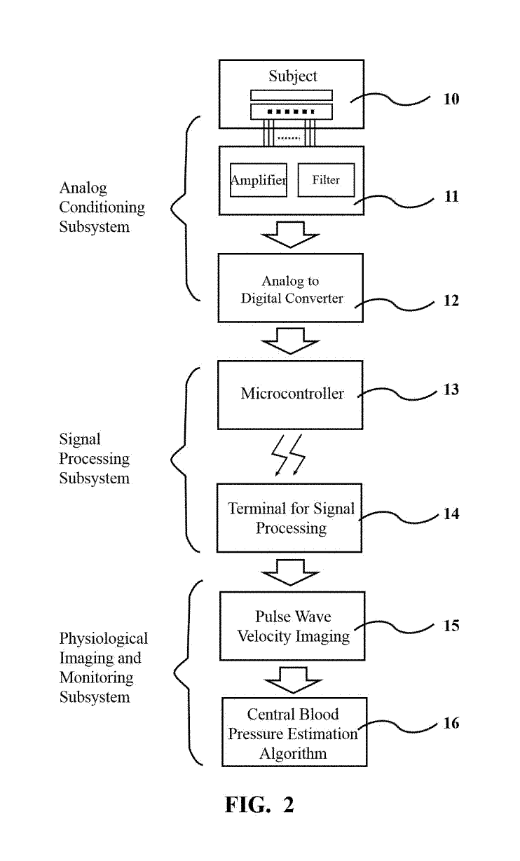

FIG. 2 shows a block diagram of the system according to an embodiment of the subject invention, including an analog conditioning subsystem, a signal processing subsystem, and a physiological imaging subsystem. The analog conditioning subsystem can include a multi-sensor acquisition unit 10 and a front-end circuit 11, which can include the amplifier and filter that process the signals to a satisfied level for the next stage. The amplified and filtered signals can then be sent to an analog-to-digital converter 12. After digitalization, signals can be transmitted to the microcontroller 13, where the signal can be processed and transmitted to the remote terminal 14 through the transmission method (e.g., Bluetooth or other wireless transmission method). A remote terminal can collect the signals and display on its monitor a real-time PWV image 15 and an estimated CBP 16. With known distances between each sensor module or node, PWV can be obtained and presented on the user interface as a PWV image. Using ECG signals from the e-textile electrodes as reference, pulse transit times (PTTs) of different body sites can be calculated, and then used to estimate the BP at different body sites. PTT has a reciprocal relationship with PWV, and can be easily obtained from two pulse signals, such as ECG and PPG signals.

With PTT calculated from the ECG and PPG from each sensor set, the spatially resolved PWV can be obtained and thereby the PWV imaging can be reconstructed (e.g., with time-space transform). Further, the CBP can be estimated through a source location method based on the PWV imaging. Systems of embodiments of the subject invention can be embedded into wearable objects (e.g., a shirt, a vest, pants) and can be used for ambulatory, clinical, and home monitoring of cardiovascular diseases (e.g., for the elderly and patients with cardiovascular risk factors), thereby providing early prediction and prevention of cardiovascular events and mortality.

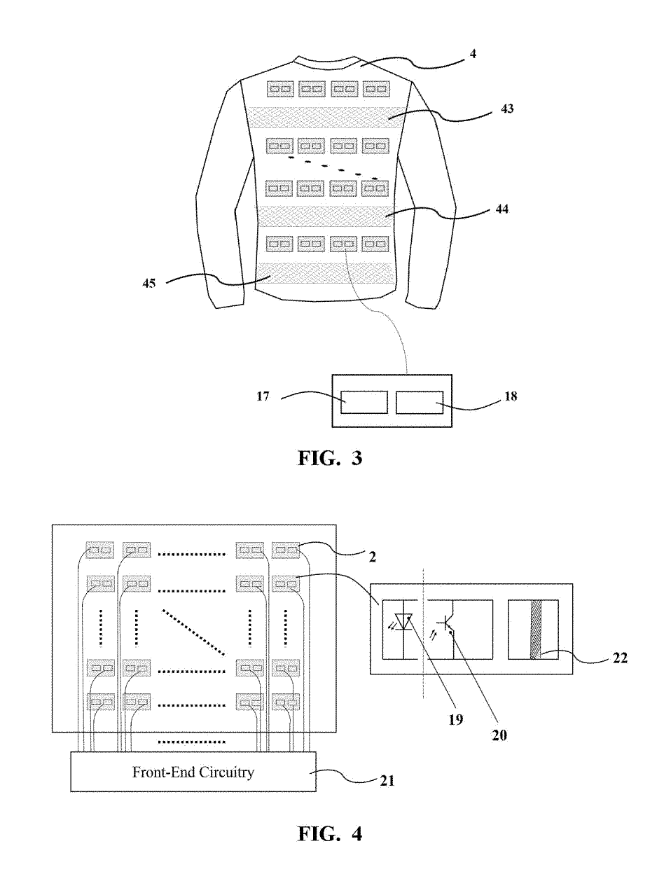

FIG. 3 is a schematic view of a signal acquisition unit. A multi-sensor pair array can be mounted as an N*M (number of rows*number of columns, where N>M, N=M, or N<M) array, with each sensor node including at least one electrode 17 and at least one PPG sensor 18 collecting ECG and PPG signals, respectively, from different sites at the same time. The e-textile electrodes 43-45 can also be integrated in the garment, so as to generate a reference ECG signal. A more detailed view of the sensor pair is provided in FIG. 4. The PPG acquisition unit can include a light source 19, such as a light emitting diode (LED) emitting light at a specific wavelength (e.g., infrared with a wavelength between 850 nanometers (nm) and 950 nm), and a photodetector 20 (e.g., a photodiode or phototransistor) that detects the reflected light. The light source 19 and the photodetector 20 can be positioned a set distance (e.g., 5 millimeters (mm)) from each other in order to achieve the best signal-to-noise ratio. In addition to the optical sensor 18, an electronic sensor 17 can be present for ECG signal acquisition. The sensor pairs can be arranged as an N*M square, with spaces in between optimized to obtain the most information and highest resolution. All N*M sensor nodes can work collectively and send their signals to the front-end circuit 21 for further processing. Though an N*M array is discussed in detail, embodiments are not limited thereto. The plurality of sensor nodes can be positioned in a circle, oval, triangle, other polygon, or in an irregular shape, for example as the specific monitoring function may require.

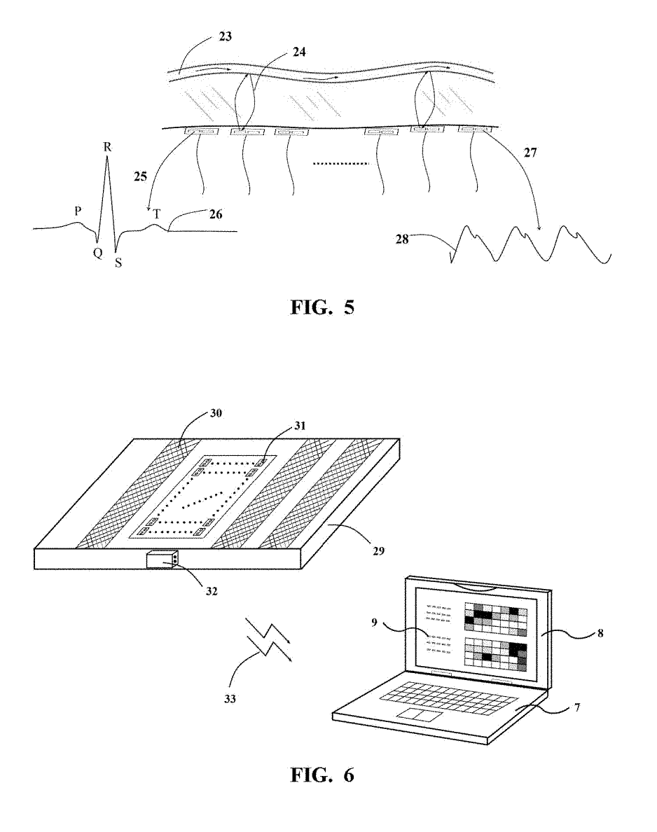

FIG. 5 shows a schematic view of the sensor pair array of FIG. 4 measuring the ECG and PPG signals of the user at different body sites. Referring to FIG. 5, the working principle of the sensing array is demonstrated. During the measurement, the electrical activity generated by the cardiac system passes through the whole body, following with the contraction of the ventricle. Then, a pressure pulse is generated that propagates through the aorta, arteries, and capillaries to the whole body and then flows back to the heart. An ECG signal 26 measures the electrical activity of heart. When at rest, each heart cell has a negative charge. As the influx of the positive ions occurs, the negative charge decreases towards zero in a process called depolarization, which will spread out orderly all through the ventricle. The ECG sensor detects and amplifies the tiny rises and falls in the voltage on the skin caused by polarization and depolarization of cardiac tissue and translates it into a waveform. A PPG sensor can be used to measure the blood volume change of one or more blood vessels. As the pressure pulse passes through the flexible vessel, the diameter of the vessel increases because of the increased volume of blood. The increased blood volume adds to the optical path of the incidence, and thus decreases the amount of reflected radiation according to Beer's Law. A photodetector detects the changes of reflected light, generates a photocurrent in response, and translates it to the PPG signal 28. A typical ECG waveform 26 of the cardiac cycle includes a P wave, a QRS complex, and a T wave, which conveys a large amount of information about the structure of the heart and the function of its electrical conduction system. A PPG waveform 28 typically includes a main peak and a smaller secondary peak. Because the electrical pulse spreads through the body near the speed of light while the pressure impulse propagates at a significantly slower speed, PTT is calculated for each heartbeat by measuring the time difference between the peak of the electrical waveform and the foot of optical waveform. PWV and BP can thus be estimated using many different methods based on PTT.

FIG. 6 shows a schematic view of a sleeping bed mounted with a multi-sensor array according to an embodiment of the subject invention. This is another unobtrusive embodiment of the multi-sensor-based PWV imaging system. ECG electrodes 30 (e.g., three ECG electrodes) can be disposed on or integrated into the bed 29 for collecting ECG signals. The multi-sensor array can include multiple sensing elements 31 and can be placed on the bed 29 (e.g., at the center of the bed 29). Each sensing unit 31 can include one or more electrical sensors and one or more optical sensors (e.g., a pair of optical sensors). The optical sensor(s) of each sensing unit 31 can work with the ECG electrodes 30 for PTT calculation when the electrical sensor(s) provide(s) the electrical potential with readings from ECG electrodes 30 as a reference. The front end circuit 32 can process the signals from the sensors, and then transmit 33 the signals (e.g., wirelessly, such as by Bluetooth) to the terminal 7.

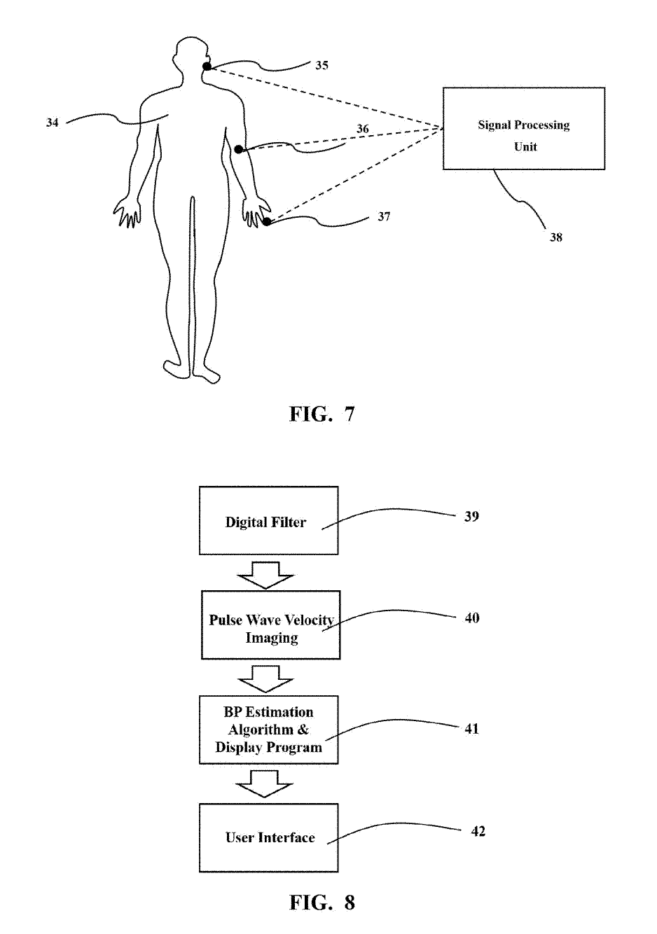

To improve the accuracy, signals from other sites of the body, for example the ear 35, arm 36, and/or finger 47, can be extracted and used as a calibration to the signals extracted from the chest and the back 34. These signals can be processed by a signal processing box 38. FIG. 7 shows a schematic view of a PPG body sensor network to improve the accuracy of central BP estimation based on a PTT method, according to an embodiment of the subject invention PPG signals are traditionally measured at the peripheral sites, such as the finger and the ear, as they have plenty of vessels that change in blood volume and that can be extracted easily and distinctively. The average of BP/PWV measured from at least two of the ear 35, arm 36, and finger 37 can serve as a reference to monitor whether the BP/PWV measured from the upper torso 34 is within a predetermined range of a correct value, and as a standard to calibrate the result after a certain period of time.

FIG. 8 shows a block diagram of a remote terminal displaying the PWV imaging, according to an embodiment of the subject invention. The collected signals can be further processed with a digital filter 39. The digital filter can have a band-pass width of, for example, between 1 Hz and 50 Hz, though embodiments are not limited thereto. After being filtered, in step 40, values of PPG signals 28 and ECO signals 26 that vary with time in all the spots can be mapped with appropriate bit colors and displayed as time-dependent PWV imaging, as well as PPG imaging and ECG imaging. Using a customized algorithm 41, the obtained PWV imaging 40 can be used to estimate CBP and/or other physiological parameters, such as heart rate, SpO₂, BP, and respiration. The physiological imaging and the parameter(s) can then be displayed in the terminal through the user interface 42.

In an embodiment, the customized algorithm 41 for CBP estimation can utilize a method of system identification, for example, the Multiple Input Single Output (MISO) with AutoRegressive eXogenous input (ARX), though embodiments are not limited thereto. With the features extracted from the PWV imaging, the central BP can be calculated through the system transfer function:

$${BP}_{n} ( t ) = {\sum }_{k = 1}^{\text{?}} {H}_{k} ( t ) {X}_{k} ( t ) + E ( n )$$

$${X}_{k} ( t ) = {\sum }_{t = 1}^{M} {\sum }_{j = 1}^{N} {u}_{i , j} ( t ) , \\ \text{?} \text{indicates text missing or illegible when filed}$$

where Hₖ(t) is the sub-system function for the k^{th} feature Xₖ(t) obtained from the element u_{i,j}(f) collected from the sensor located at (i,j), and E(n) is the exogenous factors (e.g., age, gender, etc.) relevant to the n^{th} subject.

The methods and processes described herein can be embodied as code and/or data. The software code and data described herein can be stored on one or more machine-readable media, which may include any device or medium that can store code and/or data for use by a computer system. When a computer system reads and executes the code and/or data stored on a machine-readable medium, the computer system performs the methods and processes embodied as data structures and code stored within the computer-readable storage medium.

It should be appreciated by those skilled in the art that machine-readable media (e.g., computer-readable media) include removable and non-removable structures/devices that can be used for storage of information, such as computer-readable instructions, data structures, program modules, and other data used by a computing system/environment. A machine-readable medium includes, but is not limited to, volatile memory such as random access memories (RAM, DRAM, SRAM), and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices, or other media now known or later developed that is capable of storing computer-readable information/data. Computer-readable media should not be construed or interpreted to include any propagating signals. A machine-readable medium of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

The subject invention includes, but is not limited to, the following exemplified embodiments.

### Embodiment 1

A system for pulse wave velocity (PWV) imaging, the system comprising:

a plurality of sensor units, each sensor unit including at least one electrical sensor for detecting electrocardiogram (ECG) signals of a user and at least one optical sensor for detecting photoplethysmogram (PPG) signals of said user, wherein each optical sensor includes a light source emitting light at a first wavelength and a photodetector;

a front-end circuit in operable communication with the plurality of sensor units, the front-end circuit comprising at least one amplifier and filter circuit to amplify and filter the ECG and PPG signals and at least one analog-to-digital converter to digitize the filtered ECG and PPG signals; and

at least one processor in operable communication with the front-end circuit to calculate pulse transmit time (PTT) from at least two signals out of the at least one ECG signal and the at least one PPG signal (e.g., one ECG signal and one PPG signal, or two PPG signals), thereby generating PWV imaging.

### Embodiment 2

The system according to embodiment 1, wherein the front-end circuit further comprises a microcontroller to process the signals from the at least one analog-to-digital converter and transmit the digital signals.

### Embodiment 3

The system according to any of embodiments 1-2, wherein the front-end circuit further comprises a power source.

### Embodiment 4

The system according to embodiment 1, wherein the power source comprises at least one (rechargeable) battery.

### Embodiment 5

The system according to any of embodiments 1-4, further comprising a remote terminal in operable communication with the front-end circuit and comprising a display to display PWV images based on the ECG and PPG signals obtained from the plurality of sensor units.

### Embodiment 6

The system according to embodiment 5, wherein the microcontroller wirelessly communicates with the remote terminal.

### Embodiment 7

The system according to embodiment 6, wherein the front-end circuit (e.g., the microcontroller) communicates via Bluetooth with the remote terminal Embodiment 8. The system according to embodiment 2, further comprising a remote terminal in operable communication with the microcontroller and comprising a display to display PWV images based on the ECG and PPG signals obtained from the plurality of sensor units, wherein the microcontroller transmits the signals wirelessly (e.g., via Bluetooth) to the remote terminal.

### Embodiment 9

The system according to any of embodiments 5-8, wherein the remote terminal comprises the at least one processor.

### Embodiment 10

The system according to any of embodiments 1-9, wherein the at least one processor performs a first algorithm to calculate PTT from the at least two signals out of the at least one ECG signal and the at least one PPG signal (e.g., one ECG signal and one PPG signal, or two PPG signals), thereby generating the PWV imaging, by treating each sensor unit as an individual pixel.

### Embodiment 11

The system according to any of embodiments 1-10, wherein the at least one processor performs a second algorithm to estimate central blood pressure (CBP), of a user of the system, from the obtained PWV imaging.

### Embodiment 12

The system according to embodiment 11, wherein the second algorithm comprises utilizing a method of system identification.

### Embodiment 13

The system according to embodiment 12, wherein the second algorithm comprises utilizing Multiple Input Single Output (MISO) with AutoRegressive eXogenous input (ARX).

### Embodiment 14

The system according to any of embodiments 11-13, wherein the CBP is calculated through the system transfer function:

$${BP}_{n} ( t ) = {\sum }_{k = 1}^{\text{?}} {H}_{k} ( t ) {X}_{k} ( t ) + E ( n )$$

$${X}_{k} ( t ) = {\sum }_{t = 1}^{M} {\sum }_{j = 1}^{N} {u}_{i , j} ( t ) , \\ \text{?} \text{indicates text missing or illegible when filed}$$

where Hₖ(t) is sub-system function for the k^{th} feature Xₖ(t) obtained from element u_{i,j}(t) collected from a sensor located at (i,j), and E(n) represents exogenous factors (e.g., age, gender, etc.) relevant to the n^{th} subject.

### Embodiment 15

The system according to any of embodiments 1-14, wherein the plurality of sensor units are provided in an array (e.g., an N*M array, where N is number of rows, M is number of columns, and N>M, N=M, or N<M are all valid).

### Embodiment 16

The system according to any of embodiments 1-15, wherein each light source is a light emitting diode (LED).

### Embodiment 17

The system according to any of embodiments 1-16, wherein the first wavelength is an infrared wavelength in a range of from 850 nm to 950 nm.

### Embodiment 18

The system according to any of embodiments 1-17, wherein each photodetector is a photodiode or a phototransistor.

### Embodiment 19

The system according to any of embodiments 1-18, wherein each light source is spaced apart from the photodetector of the same sensor unit by a distance of 5 mm.

### Embodiment 20

The system according to any of embodiments 1-19, wherein the at least one processor performs a third algorithm to generate ECG imaging and PPG imaging based on the ECG signals and PPG signals, respectively, detected from the sensor units.

### Embodiment 21

The system according to any of embodiments 1-20, wherein the at least one processor performs a fourth algorithm to obtain at least one of blood pressure, heart rate, SpO₂, and respiration of a user of the system.

### Embodiment 22

The system according to any of embodiments 5-21, wherein the remote terminal displays physiological parameters of a user of the system.

### Embodiment 23

The system according to any of embodiments 1-14 or 16-22, wherein the plurality of sensor units are provided in a circular arrangement.

### Embodiment 24

The system according to any of embodiments 1-14 or 16-22, wherein the plurality of sensor units are provided in an oval arrangement.

### Embodiment 25

The system according to any of embodiments 1-14 or 16-22, wherein the plurality of sensor units are provided in an irregularly-shaped arrangement.

### Embodiment 26

The system according to any of embodiments 1-25, further comprising a wearable garment in which the plurality of sensor units are provided.

### Embodiment 27

The system according to embodiment 26, wherein the front-end circuit is also provided on the wearable garment.

### Embodiment 28

The system according to any of embodiments 26-27, wherein the wearable garment is a shirt.

### Embodiment 29

The system according to any of embodiments 26-27, wherein the wearable garment is a vest.

### Embodiment 30

The system according to any of embodiments 1-25, further comprising a bed on which the plurality of sensor units are provided.

### Embodiment 31

The system according to embodiment 30, wherein the front-end circuit is also provided on the bed.

### Embodiment 32

The system according to any of embodiments 1-31, wherein each optical sensor comprises at least two light sources operating at different spectral ranges, wherein each respective photodetector PPG signals from different depths of peripheral arteries of a user of the system.

### Embodiment 33

The system according to embodiment 32, wherein the at least one processor performs a fifth algorithm to generate PWV imaging of blood vessels at different depths of sensing sites of the sensor units.

### Embodiment 34

The system according to any of embodiments 32-33, wherein the at least one processor performs a sixth algorithm to generate PPG imaging based on the PPG signals collected at different depths of sensing sites of the sensor units.

### Embodiment 35

The system according to any of embodiments 1-34, further comprising a PPG body sensor network for collecting PPG signals from different sites of a user, separate from the plurality of sensor units, thereby increasing the accuracy of PTT determination.

### Embodiment 36

The system according to embodiment 35, wherein the PPG body sensor network for collecting PPG signals from different sites of a user is configured to collect PPG signals from at least one of a brachial artery, a radial artery, a fingertip, am earlobe, and a toe.

### Embodiment 37

The system according to any of embodiments 1-36, further comprising a BP body sensor network for collecting BP readings from different sites of a user, separate from the plurality of sensor units, thereby increasing the accuracy of BP determination.

### Embodiment 38

The system according to any of embodiments 1-37, further comprising a plurality of e-textile electrodes positioned to contact a user of the system to generate a reference ECG signal to be used as a reference signal during the calculation of PTT.

### Embodiment 39

The system according to embodiment 38, comprising at least three e-textile electrodes.

### Embodiment 40

The system according to embodiment 38, comprising exactly three e-textile electrodes.

### Embodiment 41

The system according to any of embodiments 38-40, wherein the e-textile electrodes are provided in or on a surface of a wearable garment or bed having the plurality of sensor units provided thereon.

### Embodiment 42

A method of obtaining pulse wave velocity (PWV) imaging, the method comprising:

providing the system according to any of embodiments 1-41 to a user-, and

positioning the system such that the plurality of sensor units are in contact with the user, and

obtaining PWV imaging of the user by utilizing the system.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein (including those in the “References” section) are incorporated by reference in their entirety, including all figures and tables, to the extent they are not inconsistent with the explicit teachings of this specification.

### REFERENCES

1. Y. Zheng et al., “Unobtrusive Sensing and Wearable Devices for Health Informatics,” IEEE Transactions on Biomedical Engineering, Volume: 61, Issue 5, pp. 1538-1554, May 2014.
2. U.S. Pat. No. 6,475,153 B1 May 2002, Mohammad et al.
3. US 2008/0312542 A1, Matthew J. Banet et al.
4. W. Gu, C. Poon, H. Leung, M. Sy, M. Wong, and Y. Zhang, “A novel method for the contactless and continuous measurement of arterial blood pressure on a sleeping bed,” in Engineering in Medicine and Biology Society, 2009. EMBC 2009. Annual International Conference of the IEEE, 2009, pp. 6084-6086.
5. WO 2013110929 A1, T. Schaeffier et al.
6. Hatschek, R. A. D., Device and method for the measurement of blood pressure. 1992, Google Patents.
7. Zhou, Z. et al., Vital sign monitor for cufflessly measuring blood pressure without using an external calibration. 2008, Google Patents.
8. N, B. J. and F. M. J, Blood pressure measurement. 1964, Google Patents.
9. Trimmer, G. A. and E. W. Slechta, Method and apparatus for measuring blood pressure and pulse rate. 1981, Google Patents.
10. Golub, H. L., Method and apparatus for non-invasive, cuffless continuous blood pressure determination. 1999, Google Patents.
11. Asmar, R., Device for assessing cardiovascular function, physiological condition, and method thereof. 2003, Google Patents.
12. Mills, A. K., Device and method for noninvasive continuous determination of physiologic characteristics. 2003, Google Patents.
13. Baruch, M. C., D. W. Gerdt, and C. Adkins, Apparatus and method for measuring pulse transit time. 2004, Google Patents.
14. Rantala, B., Measuring blood pressure. 2006, Google Patents.
15. Zhou, Z. et al., Vital sign monitor for cufflessly measuring blood pressure without using an external calibration. 2008, Google Patents.

What is claimed is:

1. A system for pulse wave velocity (PWV) imaging, the system comprising:
a plurality of sensor units, each sensor unit including at least one electrical sensor for detecting electrocardiogram (ECG) signals of a user and at least one optical sensor for detecting photoplethysmogram (PPG) signals of said user, wherein each optical sensor includes a light source emitting light at a first wavelength and a photodetector,
a front-end circuit in operable communication with the plurality of sensor units, the front-end circuit comprising at least one amplifier and filter circuit to amplify and filter the ECG and PPG signals and at least one analog-to-digital converter to digitize the filtered ECG and PPG signals;
an object on which the plurality of sensor units and the front-end circuit are provided, wherein the object is a wearable garment or a bed; and
at least one processor in operable communication with the front-end circuit to calculate pulse transmit time (PTT) from at least two signals out of the at least one ECG signal and the at least one PPG signal, thereby generating PWV imaging.

2. The system according to claim 1, wherein the front-end circuit further comprises a power source and a microcontroller to process the signals from the at least one analog-to-digital converter and transmit the digital signals.

3. The system according to claim 1, further comprising a remote terminal in operable communication with the front-end circuit and comprising a display to display PWV images based on the ECG and PPG signals obtained from the plurality of sensor units, wherein the front-end circuit wirelessly communicates with the remote terminal.

4. The system according to claim 3, wherein the remote terminal comprises the at least one processor.

5. The system according to claim 1, wherein the at least one processor performs a first algorithm to calculate PTT from the at least two signals out of the at least one ECG signal and the at least one PPG signal, thereby generating the PWV imaging, by treating each sensor unit as an individual pixel.

6. The system according to claim 1, wherein the at least one processor performs a second algorithm to estimate central blood pressure (CBP), of a user of the system, from the obtained PWV imaging.

7. The system according to claim 6, wherein the second algorithm comprises utilizing Multiple Input Single Output (MISO) with AutoRegressive eXogenous input (ARX).

8. The system according to claim 7, wherein the CBP is calculated based on the system transfer function:
$${BP}_{n} ( t ) = {\sum }_{k = 1}^{\text{?}} {H}_{k} ( t ) {X}_{k} ( t ) + E ( n )$$
$${X}_{k} ( t ) = {\sum }_{t = 1}^{M} {\sum }_{j = 1}^{N} {u}_{i , j} ( t ) , \\ \text{?} \text{indicates text missing or illegible when filed}$$
where Hₖ(t) is sub-system function for the k^{th} feature Xₖ(t) obtained from element u_{i,j}(t) collected from a sensor located at (i,j), and E(n) represents exogenous factors relevant to the n^{th} subject.

9. The system according to claim 1, wherein the plurality of sensor units are provided in an array on the wearable garment.

10. The system according to claim 1, wherein the at least one processor performs a third algorithm to generate ECG imaging and PPG imaging based on the ECG signals and PPG signals, respectively, detected from the sensor units, and
wherein the at least one processor performs a fourth algorithm to obtain at least one of blood pressure, heart rate, SpO₂, and respiration of a user of the system.

11. The system according to claim 1, wherein each optical sensor comprises at least two light sources operating at different spectral ranges, wherein each respective photodetector collects PPG signals from different depths of peripheral arteries of a user of the system.

12. The system according to claim 11, wherein the at least one processor performs a fifth algorithm to generate PWV imaging of blood vessels at different depths of sensing sites of the sensor units.

13. The system according to claim 11, wherein the at least one processor performs a sixth algorithm to generate PPG imaging based on the PPG signals collected at different depths of sensing sites of the sensor units.

14. The system according to claim 1, further comprising at least three e-textile electrodes positioned to contact a user of the system to generate a reference ECG signal to be used as a reference signal during the calculation of PTT, wherein the e-textile electrodes are provided on the object having the plurality of sensor units and the front-end circuit provided thereon.

15. The system according to claim 1, further comprising a PPG body sensor network for collecting PPG signals from different sites of a user, separate from the plurality of sensor units, thereby increasing the accuracy of BP determination,
wherein the PPG body sensor network for collecting PPG signals from different sites of a user is configured to collect PPG signals from at least one of a brachial artery, a radial artery, a fingertip, an earlobe, and a toe.

16. The system according to claim 1, further comprising a BP body sensor network for collecting blood pressure readings from different sites of a user, separate from the plurality of sensor units, thereby increasing the accuracy of central BP determination.

17. The system according to claim 1, wherein the object having the plurality of sensor units and the front-end circuit provided thereon is a shirt or a vest.

18. A method of obtaining pulse wave velocity (PWV) imaging, the method comprising:
providing the system according to claim 17 to a user, and
positioning the shirt or vest such that the plurality of sensor units are in contact with the user, and
obtaining PWV imaging of the user by utilizing the system.

19. The system according to claim 1, wherein the object having the plurality of sensor units and the front-end circuit provided thereon is a bed.

20. A method of obtaining pulse wave velocity (PWV) imaging, the method comprising:
providing the system according to claim 19 to a user, and
positioning the bed such that the plurality of sensor units are in contact with the user; and
obtaining PWV imaging of the user by utilizing the system.
